(19) United States
(12) Patent Application Publication
(10) Pub. No.: US 2010/0074942 A1
(43) Pub. Date: Mar. 25, 2010

(54) DEVICE AND METHOD FOR INTRAOCULAR DRUG DELIVERY

(75) Inventors: Buddy D. Ratner, Seattle, WA (US); Tueng T. Shen, Redmond, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: WASHINGTON, UNIVERSITY OF, Seattle, WA (US)

(21) Appl. No.: 12/558,391

(22) Filed: Sep. 11, 2009

Related U.S. Application Data
(63) Continuation of application No. PCT/US2008/057129, filed on Mar. 14, 2008.
(60) Provisional application No. 60/894,833, filed on Mar. 14, 2007.

(51) Int. Cl. A61K 9/00; A61F 2/00; A61K 31/497; B23P 19/04

(52) U.S. Cl. 424/429; 424/427; 514/253.08; 29/282

(57) ABSTRACT

Intraocular devices having a drug delivery construct attached thereto, and methods for using the devices for intraocular drug delivery and the treatment and/or prevention of conditions.

### CROSS-REFERENCE TO RELATED APPLICATION

This application is a continuation of International Application No. PCT/US2008/057129, filed Mar. 14, 2008, which claims the benefit of U.S. Patent Application No. 60/894,833, filed Mar. 14, 2007, each application is expressly incorporated herein by reference in its entirety.

### GOVERNMENT RIGHTS

This invention was made with government support under Contract No. EEC-9529161 awarded by the National Science Foundation. The government has certain rights in the invention.

### FIELD OF THE INVENTION

The present invention relates to local therapies for the eye and, more specifically, to a device and method for intraocular drug delivery.

### BACKGROUND OF THE INVENTION

Insertion of an intraocular lens is the most commonly performed eye surgical procedure. There are approximately 10 million intraocular lenses implanted each year. Worldwide there are about 50 million people who have benefited from intraocular lens implantation. Overall, millions of eyes surgeries are performed each year.

Endophthalmitis involves inflammation of the intraocular cavities (i.e., the aqueous or vitreous humor) usually caused by infection. The most common cause of endophthalmitis is a bacterial infection after cataract surgery. It has been reported that the endophthalmitis rates from acute intraocular infection post-operation is 1/1000 in the 1990s and has grown to 1/400 more recently.

Infection (post-operative endophthalmitis) is a consistent concern, and when infection does occur, the outcome can be disastrous. Antibiotics are routinely administered locally for eye surgeries. However, the short residence time of such delivery (often via drops into the eye) requires frequent administration for effective prophylaxis-administration every four hours or more. This can lead to patient compliance problems. Also, the dose of expensive antibiotics is great, typically greater than actually required for 100% bacterial kill. The large dose is administered to compensate for overflow from the eye and to provide a high concentration during the period where the antibiotic is being diluted by tears and other body fluids. The large dose of antibiotics can also lead to toxicity to surrounding tissue.

Therefore, there is a need for a local and sustained delivery of therapeutic drug compounds, such as antibiotic and anti-inflammatory compounds, for ophthalmologic surgery.

### SUMMARY OF THE INVENTION

The present invention provides devices and methods for intraocular drug delivery.

In one aspect, the present invention provides intraocular devices having a drug delivery construct attached thereto, the drug delivery construct comprising a polymeric or hydrogel substrate having a surface with a plurality of alkyl groups covalently coupled thereto, wherein the construct includes a therapeutic drug compound.

In one embodiment, the intraocular device is a intraocular lens comprising:

(a) an optic means having an anterior and a posterior surface and a periphery;

(b) at least two resilient haptic means, the haptic means having at least one end thereof secured to the optic means with the haptic means extending outwardly from the periphery of the optic means;

(c) at least two haptic engaging means formed on the posterior surface of the optic means adjacent the periphery thereof for selectively releasably retaining the haptic means in an inwardly flexed position in proximate relation to the periphery of the optic means, the haptic engaging means being positioned for engagement with the haptic means at a position intermediate the ends thereof; and

(d) a drug delivery construct attached to at least one haptic means, the drug delivery construct comprising a polymeric or hydrogel substrate having a surface with a plurality of alkyl groups covalently coupled thereto, wherein the construct includes a therapeutic drug compound.

In another embodiment, the intraocular device is a capsular tension ring comprising:

(a) a loop formed of biocompatible material, the loop being operable to generally prevent shrinkage of the capsular bag following implantation therein; and

(b) a drug delivery construct attached to the loop, the drug delivery construct comprising a polymeric or hydrogel substrate having a surface with a plurality of alkyl groups covalently coupled thereto, wherein the construct includes a therapeutic drug compound.

In certain embodiments, the therapeutic drug compound is an antibiotic such as norfloxacin hydrochloride.

In another aspect of the invention, a method for intraocular drug delivery is provided. In one embodiment, the method includes inserting an intraocular device into an eye, the intraocular device having a drug delivery construct attached thereto, the drug delivery construct comprising a polymeric or hydrogel substrate having a surface with a plurality of alkyl groups covalently coupled thereto, wherein the construct includes a therapeutic drug compound.

In another aspect, the invention provides a method for treating and/or preventing a disease or condition, comprising introducing an intraocular device into the eye of a subject in need thereof, the intraocular device having a drug delivery construct attached thereto, the drug delivery construct comprising a polymeric or hydrogel substrate having a surface with a plurality of alkyl groups covalently coupled thereto, wherein the construct includes a therapeutic drug compound.

In one embodiment, the disease or condition is an infection.

In embodiments of the above methods, the intraocular device is an intraocular lens or capsular tension ring.

In embodiments of the above methods, the therapeutic drug compound is an antibiotic such as norfloxacin hydrochloride.

In a further aspect, the invention provides a kit for attaching a drug delivery construct to an intraocular device. In one embodiment, the kit includes:

(a) a tube with a drug delivery construct attached thereto, the drug delivery construct comprising a polymeric or hydrogel substrate having a surface with a plurality of alkyl groups covalently coupled thereto, wherein the construct includes a therapeutic drug compound; and

(b) a tool for removing the drug delivery construct from the tube to an intraocular device.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A and 1B are illustrations of a representative device of the invention, an intraocular lens (IOL) with attached drug delivery constructs;

FIGS. 1C and 1D are illustrations of representative shapes for drug delivery constructs useful in the present invention;

FIG. 2 shows a representative IOL device of the invention positioned in an eye;

FIG. 3 is an illustration of a representative device of the invention, a capsular tension ring with attached drug delivery constructs;

FIG. 4 is a graph comparing percent release (% R) and release rate (RR) of norfloxacin from drug delivery constructs used in the invention, norfloxacin-containing poly(HEMA);

FIG. 5 is a schematic illustration of a procedure for coating a polymeric substrate with an alkyl layer to provide a product useful for making a representative drug delivery construct;

FIG. 6 is a schematic illustration of a representative drug delivery construct useful in the invention;

FIGS. 7A and 7B show the electron spectroscopy for chemical analysis (ECSA) of drug delivery constructs, poly(HEMA) with (7A) and without (7B) alkyl layer coating;

FIG. 8 is a graph comparing antibiotic release profiles of representative drug delivery constructs useful in the invention;

FIGS. 9A-D are scanning electron microscope (SEM) images of the surfaces of representative drug delivery constructs, alkyl-modified poly(HEMA)s;

FIGS. 10A and 10B are graphs comparing release rate and cumulative release of antibiotic from representative drug delivery constructs useful in the invention;

FIG. 11 is a graph comparing antibiotic release from representative drug delivery constructs compared to ideal release;

FIG. 12 is a graph comparing bacteria killing (cell concentration) resulting from in vitro antibiotic release from a representative drug delivery construct useful in the invention;

FIGS. 13A and 13B are images of silicone membrane surfaces incubated without (13A) and with (13B) a representative drug delivery construct containing norfloxacin;

FIG. 14 is a photograph showing implantation of a representative intraocular lens-hydrogel construct of the invention into the eye of a rabbit post-cataract removal surgery;

FIGS. 15A and 15B are photographs comparing the eye of a control rabbit (antibiotic and steroid administered topically by drops) and the eye of an experimental rabbit (steroid administered topically by drops, antibiotic administered through a representative intraocular lens-hydrogel construct of the invention) post-cataract removal/IOL implantation surgery;

FIG. 16 is a photograph of the eye of a rabbit induced with endophthalmitis 24 hours post-inoculation;

FIG. 17 is a graph comparing norfloxacin concentration (mg/mL) over time (6 days) for rabbits having implanted representative intraocular lens-hydrogel (norfloxacin) constructs of the invention (□, Staphylococcus epidermidis (SE) challenge, no antibiotics administered topically; and Δ, no challenge) to MIC (minimum inhibitory concentration);

FIGS. 18A and 18B are photographs comparing the eye of a control rabbit (antibiotic administered topically dropwise) (18A) and the eye of an experimental rabbit (steroid administered topically dropwise, antibiotic administered through representative intraocular lens-hydrogel construct) (18B) three days post-cataract removal/IOL implantation surgery; and

FIG. 19 is an illustration of kit components for attaching a drug delivery construct to an intraocular device.

### DETAILED DESCRIPTION OF THE INVENTION

The present invention provides devices and methods for intraocular drug delivery. The device of the invention is an implantable intraocular device having an attached drug delivery construct. The construct includes one or more therapeutic drug compounds that are released over time into the eye when the device is implanted. The drug delivery construct includes a polymeric or hydrogel substrate having a surface to which a plurality of alkyl groups are covalently coupled. The intraocular devices of the invention are useful in methods for delivering one or more therapeutic compounds to the eye. The intraocular devices of the invention are also useful for preventing or treating an eye condition, such as infection, particularly, preventing or treating eye conditions post-cataract surgery.

In one aspect, the invention provides an intraocular device having a drug delivery construct attached thereto. The drug delivery construct is a polymeric or hydrogel substrate having a surface with a plurality of alkyl groups covalently coupled thereto. The drug delivery construct contains one or more therapeutic drug compounds.

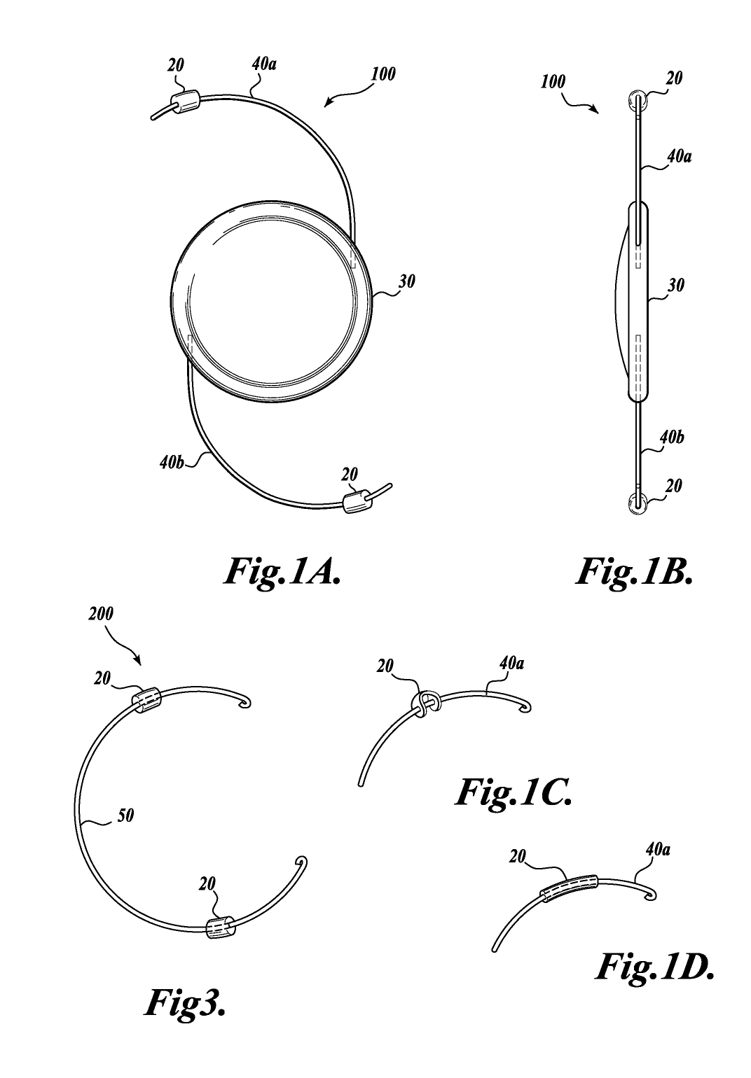

In one embodiment, the intraocular device is an intraocular lens (IOL) having one or more drug delivery constructs attached thereto (FIGS. 1A and 1B). Intraocular lenses are artificial lenses that replace the eye's natural lens that is removed during cataract surgery. An intraocular lens is an implanted lens in the eye replacing the natural crystalline lens, when, for example, the crystalline lens has been clouded by a cataract, or in refractive surgery to change the eye's optical power. IOLs are positioned in the eye using haptics, spring-like structures that immobilize the lens in the capsular bag in the eye's posterior chamber.

As shown in FIGS. 1A and 1B, the intraocular lens 100 includes optic means 30 and at least two haptics 40a and 40b. Drug delivery construct 20 is attached to at least one haptic 40a. More than one drug delivery construct 20 can be attached to the device's haptics.

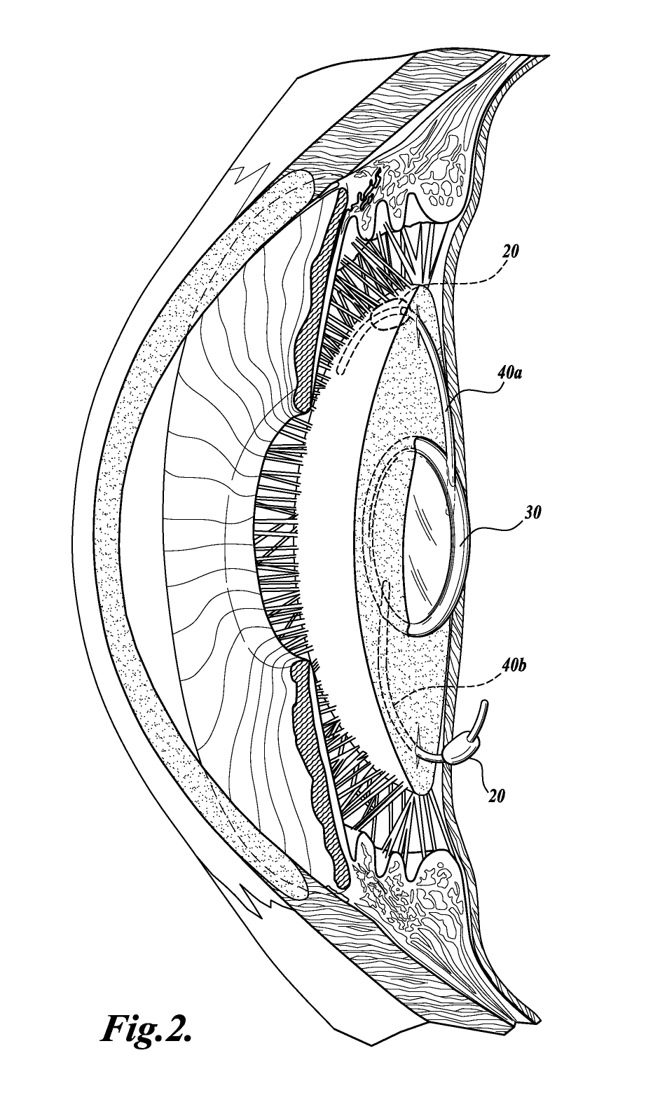

The drug delivery construct can be associated with an intraocular lens haptic during surgery. In the operating room, the surgeon can thread the drug delivery construct onto the haptic and secure the device in the capsular bag. The drug delivery construct can be threaded on (attached to) one or both haptics of the device. The device can be secured in the capsular bag to position the drug delivery construct outside the optical axis as shown in FIG. 2. The device can be removed, if needed, post-operatively. The drug delivery construct can hold sufficient quantities of therapeutic drug compounds (e.g., high potency antibiotics) to achieve release at a constant rate (zero order release) for at least one week. The release of antibiotics over time is effective in reducing the risk of infection subsequent to intraocular lens implantation.

In one embodiment, the intraocular lens of the invention includes:

(a) an optic means having an anterior and a posterior surface and a periphery;

(b) at least two resilient haptic means, the haptic means having at least one end thereof secured to the optic means with the haptic means extending outwardly from the periphery of the optic means;

(c) at least two haptic engaging means formed on the posterior surface of the optic means adjacent the periphery thereof for selectively releasably retaining the haptic means in an inwardly flexed position in proximate relation to the periphery of the optic means, the haptic engaging means being positioned for engagement with the haptic means at a position intermediate the ends thereof; and

(d) a drug delivery construct attached to at least one haptic means, the drug delivery construct comprising a polymeric or hydrogel substrate having a surface with a plurality of alkyl groups covalently coupled thereto, wherein the construct includes a therapeutic drug compound.

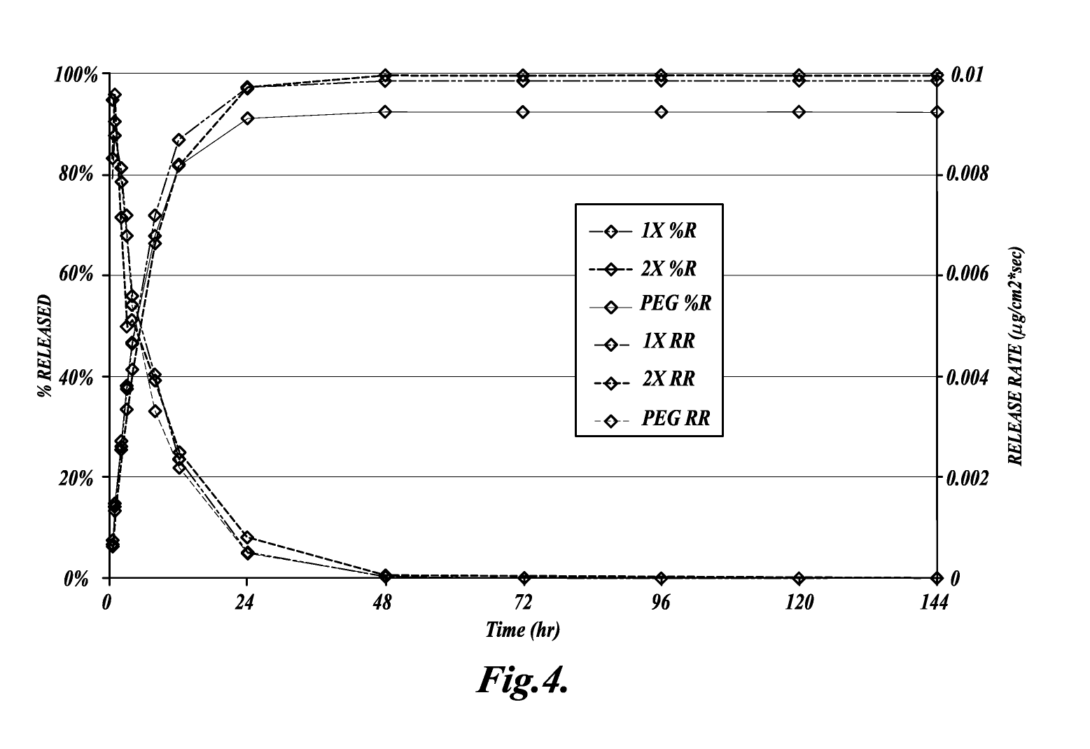

In one embodiment, the intraocular device is a capsular tension ring having one or more drug delivery constructs attached thereto (FIG. 3). The capsular tension ring (CTR) was originally introduced to reinforce the zonules in eyes with zonular dehiscence and to prevent capsular phimosis and shrinkage leading to intraocular lens decentration. Since the development of the capsular tension ring in 1993, different types of rings have been developed including the capsular edge ring, the modified capsular tension ring, the coloboma ring, and the aniridia rings. As used herein, the term “capsular tension ring” refers to the capsular edge ring, the modified capsular tension ring, the coloboma ring, and the aniridia rings.

A capsular tension ring is an open ring, or an open loop, having a diameter larger than the eye's capsular bag. A capsular tension ring effectively stabilizes the capsular bag by exerting a mild centripetal pressure equally balanced all over the equatorial region of the bag. The capsular tension ring appears to be a safe and efficacious device that improves the outcome of cataract surgery when the stability of the capsular bag is compromised.

In one embodiment, the intraocular device of the invention is a capsular tension ring having one or more drug delivery constructs attached thereto. FIG. 3 is an illustration of a representative intraocular device of the invention, a capsular tension ring having two drug delivery constructs attached thereto. Referring to FIG. 3, representative intraocular device 200 includes capsular tension ring 50 and two drug delivery constructs 20.

In one embodiment, the capsular tension ring of the invention includes:

(a) a loop formed of biocompatible material, the loop being operable to generally prevent shrinkage of the capsular bag following implantation therein; and

(b) a drug delivery construct attached to the loop, the drug delivery construct comprising a polymeric or hydrogel substrate having a surface with a plurality of alkyl groups covalently coupled thereto, wherein the construct includes a therapeutic drug compound.

Depending on the needs, one or more drug delivery constructs can be attached to the intraocular device of the invention, for example, through a haptic of an intraocular lens or a capsular tension ring. When multiple drug delivery constructs are attached to an intraocular device, each drug delivery construct may contain the same or different therapeutic drug compound based on the needs of the subject being treated. Therefore, simultaneous delivery of more than one therapeutic drug compound can be achieved through the practice of the invention.

The drug delivery construct useful in the intraocular devices of the invention can have a variety of shapes and sizes. As illustrated in FIGS. 1A, 1B, 1C, and 1D, representative drug delivery constructs can be in the shape of a cylinder or ring (1A, 1B, 1D) or a disk (1C). It will be appreciated that the shape of the drug delivery construct is not critical and that any construct capable of being attached to an intraocular device, regardless of its shape, is within the scope of the invention. In one embodiment, the drug delivery construct has a diameter of from about 0.5 mm to about 3 mm. In one embodiment, the drug delivery construct has a diameter of from about 1 mm to about 2 mm.

The drug delivery construct useful in the devices and methods of the invention includes a polymeric or hydrogel substrate having a surface to which a plurality of alkyl groups are covalently coupled. The construct includes one or more therapeutic drug compounds that are released from the construct over time.

The plurality of alkyl groups covalently coupled to the polymeric or hydrogel substrate surface form a coating on the substrate surface. In one embodiment, the plurality of alkyl groups form a layer or monolayer on the substrate surface.

A variety of polymers are useful for making the substrate. Representative examples of synthetic polymers useful for making the substrate of drug delivery construct include (poly)urethane, (poly)carbonate, (poly)ethylene, (poly)propylene, (poly)lactic acid, (poly)galactic acid, (poly)acrylamide, (poly)methyl methacrylate, and (poly)styrene. Useful natural polymers include collagen, hyaluronic acid, and elastin.

A variety of therapeutic drugs can be incorporated into the construct. The therapeutic drug compound incorporated into and released from the construct can be only one of a variety of therapeutic compounds including antibiotic compounds, anti-inflammatory compounds, ophthalmic beta-blockers, carbonic anhydrase inhibitors, alpha-agonists, miotics, and prostaglandin analogs, among others.

In one embodiment, the therapeutic drug compound can be incorporated into drug delivery construct during the process of making the substrate. For example, norfloxacin (an antibiotic) was added to a solution of water and polyethylene glycol. To this solution was added 2-hydroxyethyl methacrylate (2-HEMA) and triethylene-glycol dimethylacrylate (TEGDMA) (a crosslinking agent) to provide a mixture that was then cast between two glass plates. Polymerization (24 hours) provided a gel-like substrate loaded with norfloxacin. The substrate loaded with the antibiotic was then soaked for 4 hours in distilled water, changing to new water every hour. The polymeric substrate was obtained by punching the resulting antibiotic loaded substrate into 1 cm disks.

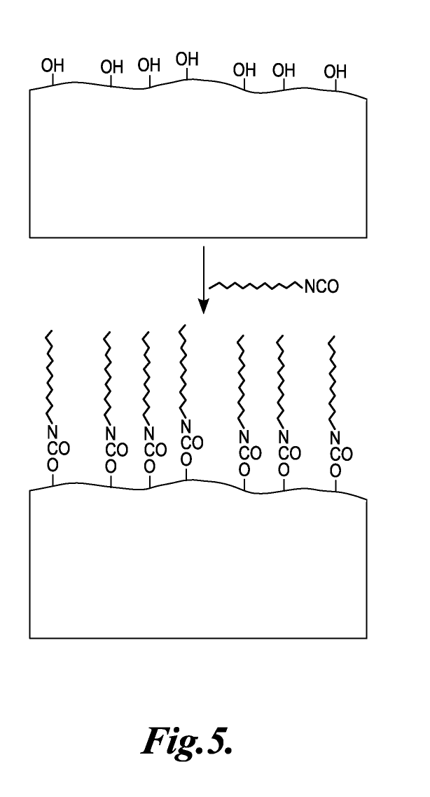

Several types of drug delivery constructs were obtained using the method described above by varying the amount of crosslinking agent and polyethylene glycol. The release of norfloxacin from representative poly(HEMA) substrates is illustrated in FIG. 4. In FIG. 4, “% R” refers to % Released, “RR” refers to Release Rate, “1X” refers to a substrate having a first amount of crosslinker (2.6% by weight), “2X” refers to a substrate having double the amount of crosslinker (5.1%), and “PEG” refers to a substrate prepared with 100 mg PEG (MW 3400 Da) added to the mixture. From the release data, the crosslinker amount did not change release performance. While not wishing to be bound by theory, it is believed that the amount of the crosslinker does not significantly affect the amount of substrate swelling.

In order to achieve the sustained and controlled drug release, the surface of the substrate is coated with a layer of alkyl groups (e.g., unbranched alkyl groups). In one embodiment, the alkyl groups are C₁₀ to C₂₂ unbranched alkyl groups. In one embodiment, the alkyl groups are C₁₂ to C₁₈ unbranched alkyl groups.

As used herein, the term “layer” refers to a layer formed by covalently attaching compounds having alkyl groups (e.g., C₁₀ to C₂₂ unbranched alkyl groups) to a polymeric or hydrogel substrate. The groups that form the layer may or may not be evenly distributed throughout the layer. The surface of the substrate to which the layer is attached may not be uniform and, as a result, the groups within the layer may not have the same height relative to each other. Moreover, the layer may extend into the substrate in the portion of the substrate close to the substrate surface. Typically, the surface layer does not penetrate the substrate surface to a depth of greater than 1 μm.

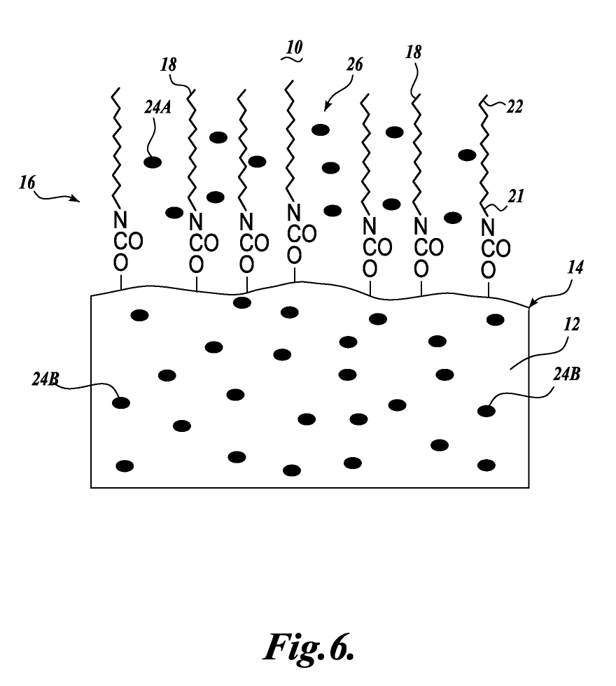

As illustrated schematically in FIG. 5, and described in Examples 1 and 2, in one embodiment of the invention, octadecyl (C₁₈) isocyanate was reacted with surface hydroxyl groups of a crosslinked poly(2-hydroxyethyl methacrylate) (poly(HEMA)) substrate loaded with a therapeutic drug compound to provide a drug delivery construct having a hydrophobic alkyl layer. The reaction was carried out in anhydrous atmosphere and was catalyzed by dibutyltin dilaurate.

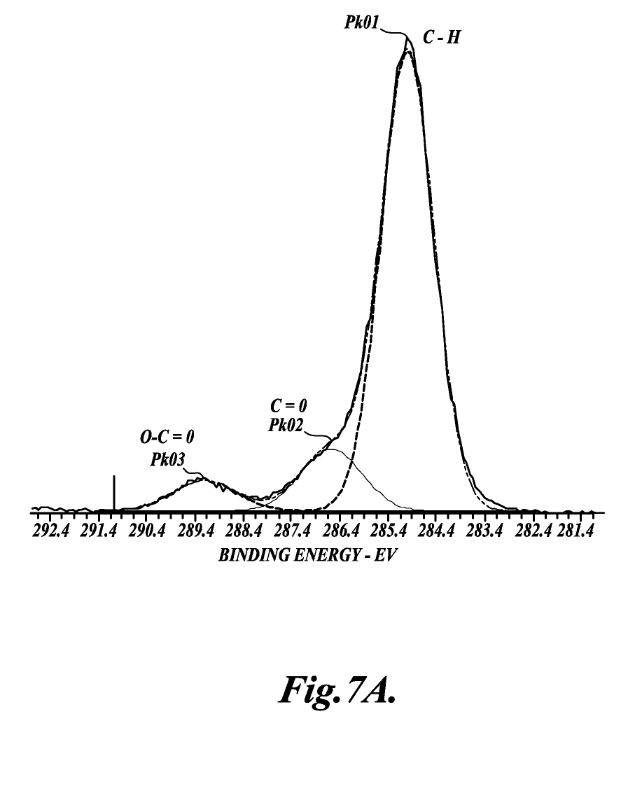

A schematic illustration of a representative drug delivery construct of the invention is shown in FIG. 6. Referring to FIG. 6, drug delivery construct 10 includes substrate 12 (made from a polymer or hydrogel) having surface 14 and surface layer 16 including a multiplicity of unbranched alkyl groups 18 (C₁₂ molecules in the embodiment shown in FIG. 6). Each alkyl group 18 includes a proximal end 21 and a distal end 22. Proximal end 20 of each alkyl group 18 is covalently coupled to substrate 12 by a urethane bond. In the embodiment shown in FIG. 6, construct 10 includes therapeutic drug compounds 24B disposed within substrate 12 and therapeutic drug compounds 24A disposed in spaces 26 intermediate alkyl groups 18 of layer 16. When the drug delivery construct is prepared from a polymerizing solution containing a therapeutic drug, the construct includes primarily therapeutic drug compounds 24B. When the drug delivery construct is prepared by soaking a substrate having an alkyl layer (as illustrated in FIG. 5), the construct includes therapeutic drug compounds 24A and 24B.

Typically, alkyl groups 18 of layer 16 are aligned side-by-side (such as shown in FIGS. 5 and 6), although the density of alkyl groups 18 may vary over polymeric substrate surface 14, and alkyl groups 18 may not be vertically aligned with respect to substrate surface 14, but may be covalently attached to their point of attachment at an angle, such as an angle of approximately 33 degrees. When substrate 12 is sufficiently porous to permit penetration of alkyl groups 18, then layer 16 can extend into the portion of substrate 12 adjacent to substrate surface 14. As noted above, layer 16 does not typically penetrate more than 1 μm into substrate 12 (i.e., typically few or no alkyl groups 18 penetrate further than 1 μm from substrate surface 14 into substrate 12).

The drug delivery construct useful in the invention has been described above as a polymeric or hydrogel substrate having a surface with a plurality of alkyl groups covalently coupled thereto, wherein the construct includes one or more therapeutic drug compound.

The drug delivery construct useful in the invention can also be described as including:

(a) a polymeric or hydrogel substrate comprising one or more therapeutic drug compounds; and

(b) a surface layer comprising a multiplicity of alkyl groups, wherein:
(i) the multiplicity of alkyl groups defining a multiplicity of spaces therebetween; and
(ii) each member of the multiplicity of alkyl groups has a proximal end and a distal end, the proximal end covalently linked to the substrate.

In one embodiment, the therapeutic drug compounds are disposed within the substrate and also disposed in the spaces between the alkyl groups.

Representative drug delivery constructs of the invention can be prepared as described in U.S. Pat. No. 6,444,217, incorporated herein by reference in its entirety.

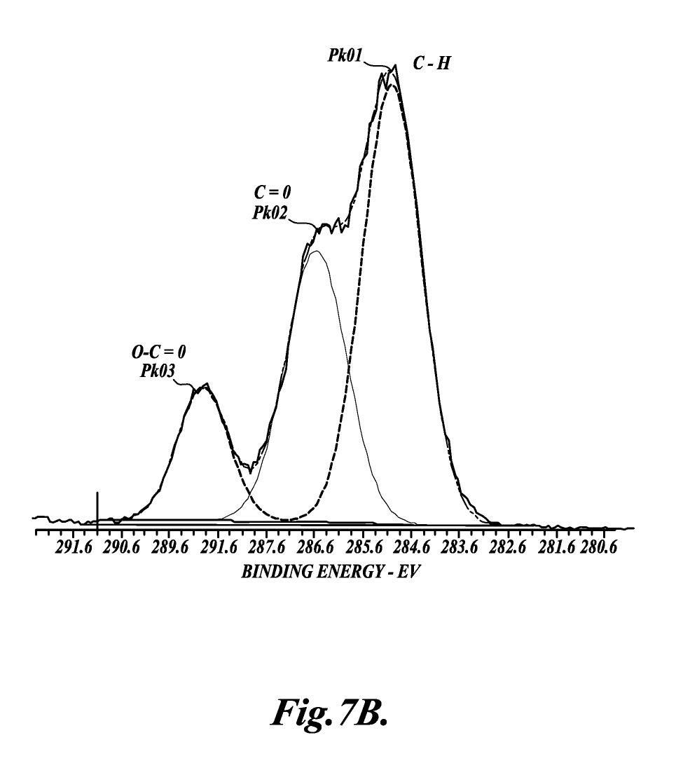

As noted above, it is believed that the constructs' alkyl layer enhances advantageous therapeutic drug release rates. To obtain drug delivery constructs having different release rates, the drug loaded poly(HEMA) substrate was reacted with octadecyl (C₁₈) isocyanate, as described herein, for varying lengths of time (e.g., 15, 30, 45, and 60 minutes). The electron spectroscopy chemical analysis (ECSA) for coated and uncoated poly(HEMA) drug delivery constructs (FIGS. 7A and 7B, respectively) indicates that there are only carbon and oxygen are present in the uncoated samples (7B), and that nitrogen is present in the coated samples (7A) due to C₁₈-isocyanate modification as indicated by the increase of C—H relative to the other bands.

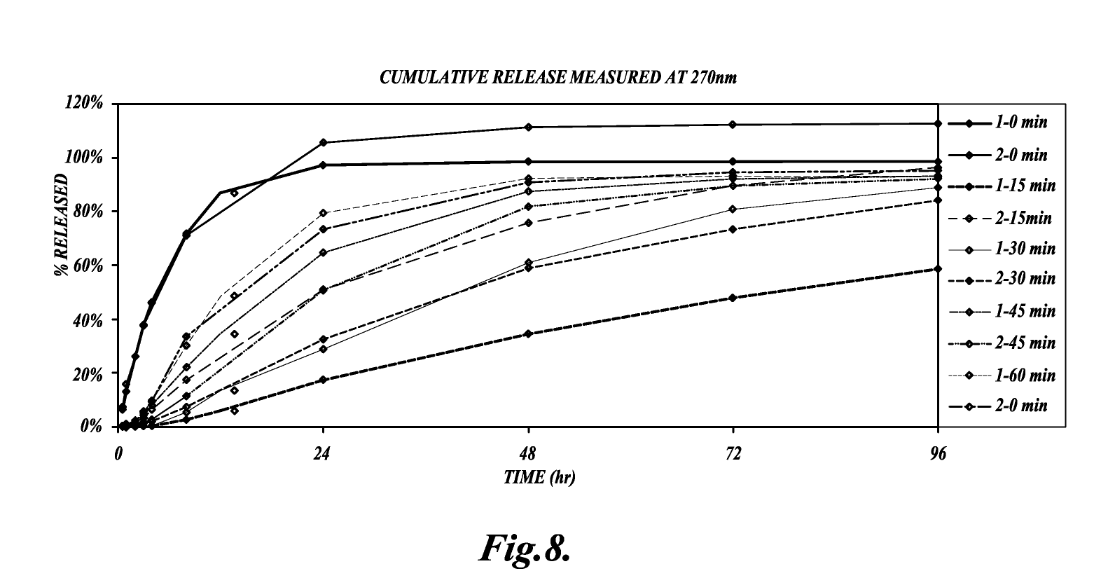

Surface coating is believed to be a factor in achieving sustained release. The drug delivery constructs prepared as described above were subjected to the antibiotic release. As illustrated in FIG. 8, the antibiotic release profiles for drug delivery constructs show a clear trend. The 60 minute (longest) coating reaction produced the most rapid release, and the 15 minute (shortest) coating reaction produced the slowest and most steady release.

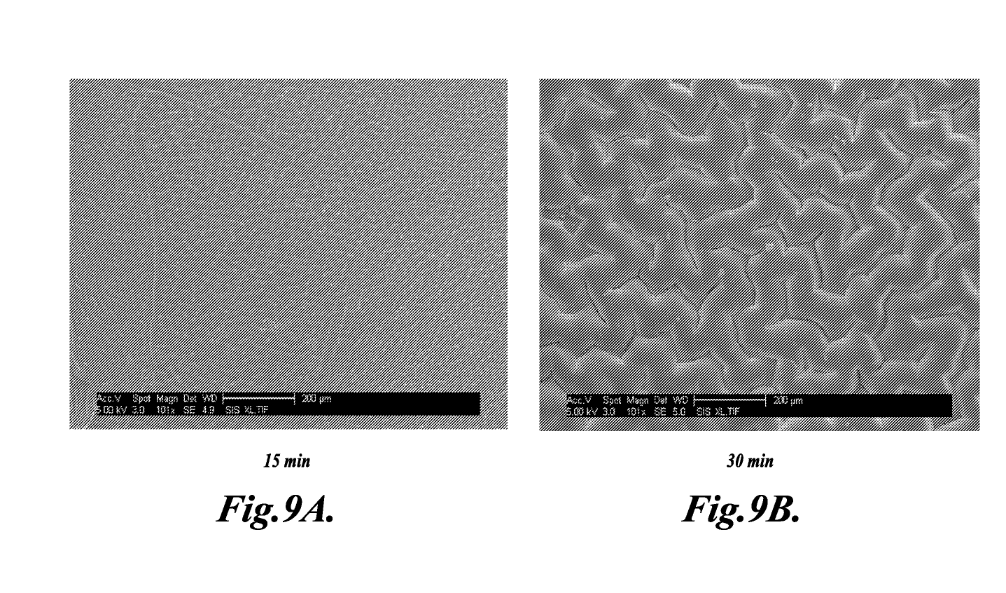

Scanning electron microscope (SEM) images of the surfaces of the drug delivery constructs, prepared as described above, are shown in FIGS. 9A-9D (C₁₈-isocyanate reaction times of 15, 30, 40, and 60 minutes, respectively). As demonstrated in FIGS. 9A-9D, with the reaction time increasing from 15 minutes (FIG. 9A) to 60 minutes (FIG. 9D), the poly(HEMA) surface was penetrated by the isocyanate reaction (FIGS. 9C and 9D), creating holes and pores through which the therapeutic drug escapes, which may be the reason that longer reaction times yield greater release.

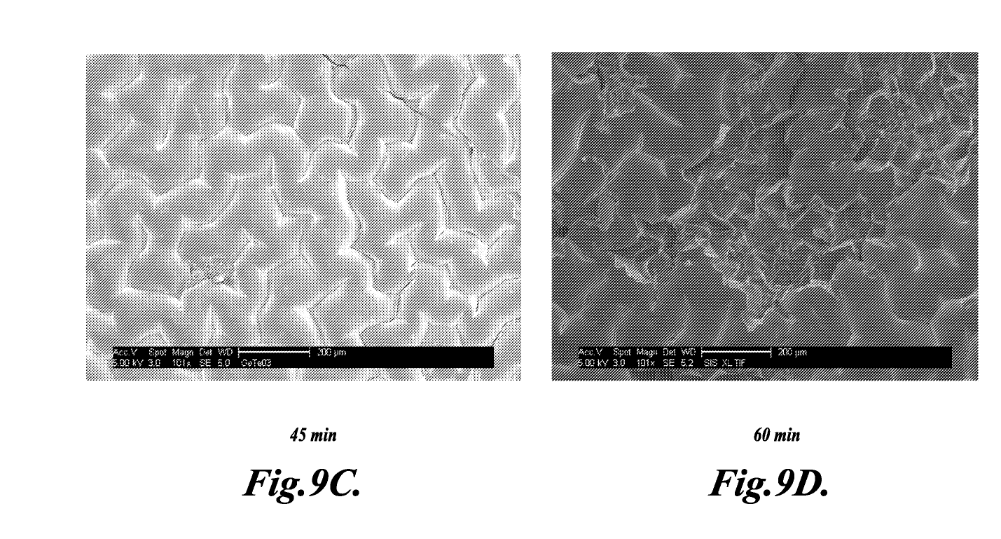

The cumulative release and release rate for the drug delivery constructs coated with alkyl groups, prepared as described above, are shown in FIGS. 10A and 10B, respectively. While some 15 minute-coated constructs show steady release, the results obtained from constructs after 30 minutes of coating are the most repeatable, and achieve acceptable release profiles. The theoretically needed flux to achieve minimum inhibitory concentration 50 (MIC50) continuously for 1 week is 9.5×10⁻⁵. This is based on volume of the anterior chamber and its fluid turnover rate, and the MIC50. The release achieved in the embodiments of the present invention is much higher than the theoretical requirement.

Because of the presence of a hydroxyl group in the side chain of the polymer, various modifications of poly(HEMA) using its primary alcohol are possible and provide a wide range of poly(HEMA) derivatives useful for making the substrates, and are described for example in Montheard, J.-P., et al., “Homopolymers and Copolymers of 2-Hydroxyethyl Methacrylate for Biomedical Applications,” Reza, A., ed., American Chemical Society, Washington D.C., 1997; pp. 699-711. A more complete overview of isocyanate chemistry (useful for attaching C₁₀ to C₂₂ unbranched alkyl molecules to a hydroxyl group on poly(HEMA) or other polymer or hydrogel) is described in Arnold, R. G. et al., Chem. Rev. 57:47-76, 1957, and in Saunders, J. H., et al., Chem. Rev. 43:203-218, 1948, incorporated herein by reference in its entirety.

Alkyl groups can be attached to the substrate by any suitable reaction. For example, the following pairs of reactive groups (each member of the pair being present on either substrate or proximal end of alkyl molecule) can be utilized to bond alkyl molecules to the substrate: hydroxyl/carboxylic acid to yield an ester linkage; hydroxyl/anhydride to yield an ester linkage; and hydroxyl/isocyanate to yield a urethane linkage. Substrates that do not possess useful reactive groups can be treated with radio-frequency discharge plasma etching to generate reactive groups (e.g., treatment with oxygen plasma to introduce oxygen-containing groups; treatment with propyl amino plasma to introduce amine groups).

The amount of therapeutic drug compound incorporated in the drug delivery construct of the invention can be varied based on the need of the subject to be treated. The drug load can be readily determined by routine experimentation. The following table lists the representative calculation of potential drug load based on substrate size (e.g., height, radius, surface area (sa), and volume).

TABLE 1
Calculation of potential therapeutic drug compound load.

| Height | Rad | SA | Vol | SA/ | amt of drug |
|---|---|---|---|---|---|
| (cm) | (cm) | (cm²) | (cm³) | vol | (mg) |
| 0.5 | 0.1 | 0.377 | 0.0157 | 24 | 186.9 |
| 0.5 | 0.083 | 0.303 | 0.0107 | 28.15 | 128.1 |
| 0.302 | 0.1 | 0.253 | 0.0095 | 26.61 | 113.1 |
| 0.3 | 0.1 | 0.251 | 0.0094 | 26.67 | 112.2 |
| 0.3 | 0.15 | 0.424 | 0.0212 | 20 | 252.3 |
| 0.296 | 0.1 | 0.249 | 0.0092 | 26.76 | 110.7 |
| 0.35 | 0.1 | 0.283 | 0.0109 | 25.71 | 130.8 |
| 0.4 | 0.1 | 0.314 | 0.0125 | 25 | 149.5 |
| 0.4 | 0.09 | 0.277 | 0.0101 | 27.22 | 121.1 |
| 0.45 | 0.09 | 0.305 | 0.0114 | 26.67 | 136.3 |
| 0.45 | 0.08 | 0.266 | 0.0090 | 29.44 | 107.7 |

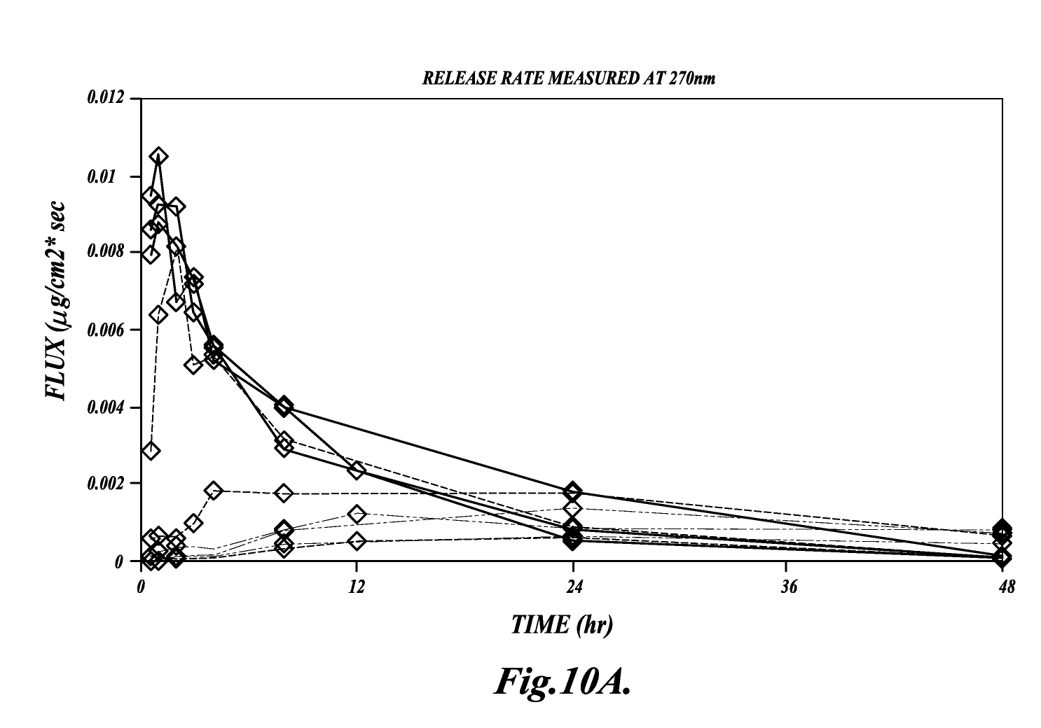

The release of the antibiotic norfloxacin from the drug delivery construct was tested. The construct, 1 cm norfloxacin-loaded poly(HEMA), was allowed to shake in water/PBS solution for one week. The construct was placed into a new solution at pre-determined time points. The amount of drug released into solution was determined by UV-Vis spectroscopy by measuring absorbance at λ=270 nm. Calculations were carried out to determine cumulative release and release rate, and the results are presented in FIG. 11.

FIG. 11 illustrates norfloxacin release rate from cylinder-shaped drug delivery constructs. The constructs were obtained after alkyl coating reaction times of 0 minutes, 15 minutes, and 30 minutes. Compared to the idea release, the drug constructs obtained by 30 minutes reaction demonstrate a steady release of norfloxacin comparable to the ideal release profile.

The drug delivery constructs were tested in vitro for its antibacterial activity. The constructs were tested in a study lasting 24 hours. The ability of an antibiotic-loaded poly(HEMA) was tested for its ability to kill bacteria both in solution and on a silicone membrane.

Staphylococcus epidermidis was grown in Tryptic Soy Broth in a 48-well plate for 24 hours. Staphylococcus epidermidis was the chosen bacteria because it is the most prevalent bacteria found in an endophthalmitis infection. The drug delivery construct and a 6 mm silicone membrane were both soaked in a culture solution. Silicone is noted to be a surface that encourages significant bacteria adhesion, especially for clinical endophthalmitis isolates. A similarly shaped and sized poly(HEMA) disk without the drug was used as the control.

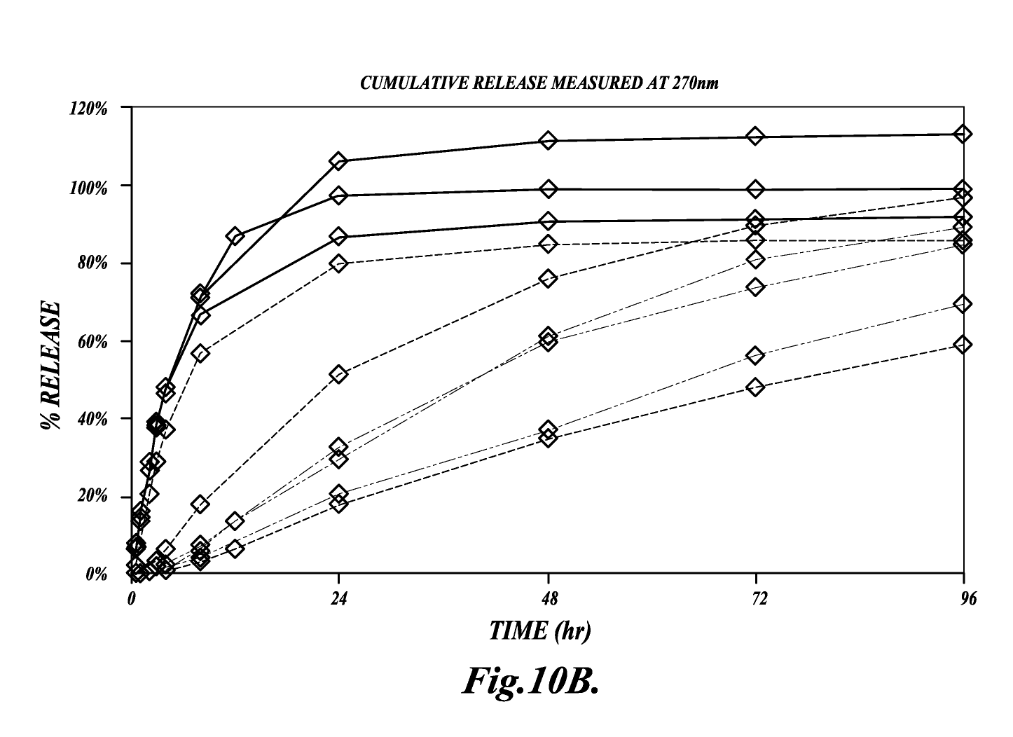

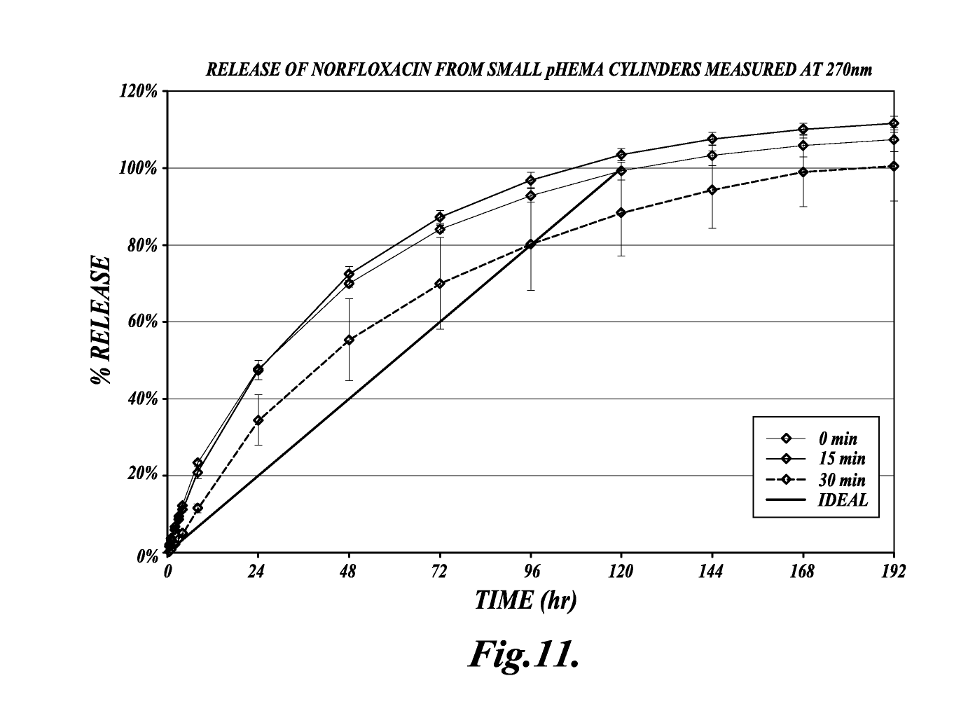

The test results are illustrated in FIG. 12. Photographs of the surfaces of the control and norfloxacin treated silicone membranes after 24 hours were taken (FIGS. 13A and 13B, respectively). After 24 hours, there are virtually no live cells adhered to the silicone membrane when norfloxacin treatment is applied (FIG. 13B), compared to the control in which there are still significant member of live cells (FIG. 13A).

In vivo test results for representative intraocular drug delivery devices of the invention are described in Examples 4 and 5.

In another aspect, the invention provides a method of intraocular drug delivery. In one embodiment, the method includes inserting into an eye an intraocular device of the invention having a drug delivery construct attached thereto.

For delivery of antibiotics and other drugs locally to implanted intraocular devices, the drug delivery construct can be attached to an intraocular device or other fixation device by the surgeon in the operating room immediately prior to insertion in the eye.

The drug delivery method of the invention has an advantage over other approaches to antibiotics delivery used in conjunction with intraocular lens surgery. The antibiotic is delivered locally to what may become the locus of the infection, the lens itself. Thus, high local doses can be realized without having to massively does other surrounding tissues. In addition, other drugs may also be loaded onto the substrate depending on need. For example, anti-inflammatory agents can be combined with antibiotics in the substrate to achieve the simultaneous treatment of inflammation and infection. Accurate dosing is ensured because the complete dose is within the construct.

Thus, in a further aspect, the invention provides a method of treating and/or preventing a disease or eye condition that includes introducing an intraocular device of the invention into the eye of a subject in need thereof.

The methods of the invention are useful for localized and controlled delivery of variety of therapeutic agents. By way of representative example, proteins, peptides, nucleic acids, insulin, estrogens, androgens, cancer chemotherapeutics, hypnotics, anti-psychotics, narcotics, diuretics and other blood-pressure-regulating drugs can be delivered using the devices of the invention.

In another aspect, the invention provides a kit for attaching a drug delivery construct to an intraocular device to provide an intraocular device of the invention having a drug delivery construct attached thereto. In one embodiment, the kit includes:

(a) a tube having a drug delivery construct attached thereto, the drug delivery construct comprising a polymeric or hydrogel substrate having a surface with a plurality of alkyl groups covalently coupled thereto, wherein the construct includes one or more therapeutic drug compounds; and

(b) a tool for removing the drug delivery construct from the tube to an intraocular device.

In one embodiment, the tube is a syringe or syringe needle. In one embodiment, the tool is a forceps.

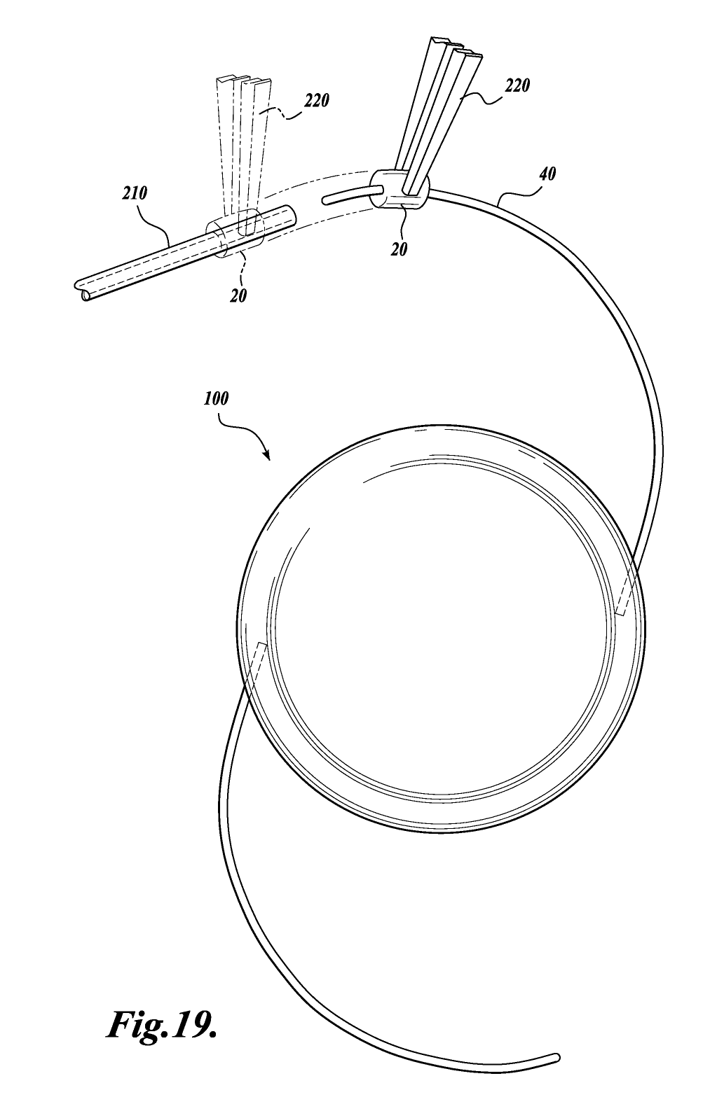

Referring to FIG. 19, tube 210 with attached drug delivery construct 20 receives the terminus of haptic 40 of intraocular device 100 (an intraocular lens). Drug delivery construct 20 is then slid from tube 20 onto haptic 40 using forceps 220. As noted above, for delivery of antibiotics and other therapeutic drug compounds locally to implanted intraocular devices, the drug delivery construct can be attached to an intraocular device or other fixation device by the surgeon, as described above, in the operating room immediately prior to insertion in the eye.

The following examples are provided for the purpose of illustrating the invention.

### EXAMPLES

### Example 1

### Materials and Methods

2-Hydroxyethyl methacrylate (HEMA, No. 04675) monomer with a purity of more than 99.5% and tetraethylene glycol dimethacrylate (TEGDMA, No. 02654) were purchased from Polysciences Inc., Warrington, Pa. Ethylene glycol (No. 32,455-8), sodium metabisulfite (No. 16,151-9), ammonium persulfate (No. 24,861-4), anhydrous tetrahydrofuran (THF, No. 40,175-7), dodecyl isocyanate (C₁₈ isocyanate), and dibutyltin dilaurate (No. 38,906-4) were received from Aldrich, Inc. All chemicals were used as received. Glass plates and glass apparatus for synthesis were soaked in 2% RBS-35 detergent (No. 27950, Pierce) overnight and rinsed with Millipore purified water prior to the experiments.

Preparation of a Polymeric Substrate (Poly(HEMA))

Crosslinked hydrogel slabs were synthesized from HEMA. Briefly, 0.5 g of 2-HEMA monomer and 0.2 g of the TEGDMA crosslinking agent were added to a mixed solution of norfloxacin and water/ethylene glycol (1 g/1.5 g) with 1 mL of 15% sodium metabisulfite and 40% ammonium persulfate as redox initiators to begin the radical polymerization. The mixture was allowed to polymerize between two clean glass plates with a Teflon gasket of thickness 0.025 in. Although the gel set within an hour, the film was allowed to stand overnight. The poly(HEMA) film was released from the glass plates and soaked in distilled water for a few days to leach out unreacted monomers, initiators, and oligomer residues. To speed the leaching process, later films were soaked in water for only 1 day. After leaching, the poly(HEMA) film was cut into smaller specimens for surface modification with C₁₈ isocyanate. The poly(HEMA) samples must be vacuum-dried prior to surface derivatization because water molecules easily terminate the urethane-linkage reaction between the hydroxyl group on the poly(HEMA) surface and the isocyanate of the C₁₂ compound.

Preparation of Alkyl Layer on Poly(HEMA) Substrate

The procedure of coating the substrate with alkyl layer is illustrated in FIG. 6. In a three-necked round-bottom flask connected to a nitrogen gas line, 4.5 mL of C₁₈ isocyanate (5%) and 0.18 mL of dibutyltin dilaurate (as the catalyst, 0.3%) were added to 90 mL of anhydrous tetrahydrofuran (THF) containing the dry polymer films. In this case, the choice of the reaction medium is important. THF is a poor swelling solvent for poly(HEMA) which prevents polymer swelling and optimizes the surface immobilization reaction of hydrocarbon chains to the gel slab. To further optimize the reaction conditions, temperature and reaction time were studied. The reaction was performed under a nitrogen atmosphere at 40, 50, or 60° C. in an oil bath. At each temperature, the reaction was allowed to run for 5, 15, 30, 45, and 60 min. At each time point, one poly(HEMA) sample was retrieved from the reaction flask and sonicated (43 kHz, L&R model T21) in fresh THF for 5 min to remove physically adsorbed C₁₈ isocyanate. Following sonication, the surface-derivatized films were blown dry with nitrogen for surface characterization.

Surface Characterization of Drug Delivery Construct

The drug delivery constructs were examined by a number of surface characterization techniques. XPS was used to measure the chemical composition and functional groups of alkyl layer. TOF-SIMS to study the molecular fragments that were chemically bonded to substrate surface. FTIR-ATR to investigate the chain order and crystalline structure, and polarized ATR to estimate the molecular chain orientation of layer.

X-Ray Photoelectron Spectroscopy

XPS, also known as electron spectroscopy for chemical analysis (ESCA), was performed with an S-Probe surface analysis system (Surface Science Instruments, Mountain View, Calif.) using a monochromatic Al K_{∝12} X-ray source to stimulate photoemission. The system consists of a 30° solid angle acceptance lens, a hemispherical analyzer, and a position-sensitive detector. All polymer samples were analyzed at a 55° takeoff angle, probing the uppermost 50-80 Å of the surface. The takeoff angle was defined as the angle between the surface normal and the axis of the analyzer acceptance lens. Survey scans (0-1000-eV binding energy) were run at an analyzer pass energy of 150 eV (resolution 4) with an X-ray spot size of 1000-1700 μm to determine the elemental composition of each surface. High-resolution O (1 s), C (1 s), and N (1 s) scans were obtained at a pass energy of 50 eV (resolution 2). The high-resolution spectra were resolved into individual Gaussian peaks using a least-squares fitting routine in the SSI software. The chemical composition of each surface was determined from the peaks resolved in the high-resolution scans. All binding energies (BEs) were referenced by setting the maximum of the resolved C (1 s) peak, corresponding to carbon in a hydrocarbon environment (CHx), to 285.0 eV. When the binding energy referencing was performed in the same manner, the primary O (1 s) peak was found to be shifted to 532.8 eV, the expected value for oxygen in an ether environment in polymers. A 5-eV electron flood gun was used to minimize surface charging. Typical pressures in the analysis chamber during spectral acquisition were 10⁻⁹ Torr.

### Example 2

### The Preparation and Characterization of a Representative Drug Delivery Construct: Octadecyl Isocyanate Surface Layer Formed on a Poly(HEMA) Substrate

As illustrated schematically in FIG. 5, surface hydroxyl groups on the poly(HEMA) were, in a concerted fashion, catalyzed to form urethane bonds with the available isocyanate groups on octadecyl isocyanate as these alkyl compounds self-assembled on the surface of the hydrogel to form surface layer.

XPS and TOF-SIMS Analysis

A typical C (1 s) XPS Spectra (FIG. 7A) of C₁₈ surface layer on poly(HEMA) substrate with different reaction times at 60° C. showed an increase of the CHₓ (methylene) and the disappearance of C—OH/CO peaks which is indicative that hydroxyl groups in poly(HEMA) are reacting with the isocyanate. Further evidence for the desired reaction was the broadening of the O—C═O peak (compared to the control reaction containing C₁₈ isocyanate but no catalyst), presumably due to its conversion into the urethane bond.

A typical O (1 s) XPS Spectra of C₁₈ surface layer on poly(HEMA) substrate from the samples described above showed that the broad, but symmetrical, O (1 s) peak observed in poly(HEMA) is split into two peaks represented by the two types of oxygen present in the urethane bond.

Normalized peak intensities of various representative negative molecular ions (via TOF-SIMS) originating from poly(HEMA) during various reaction times showed that the major moieties from poly(HEMA) disappear during the reaction within 30 min. This is consistent with XPS analysis. Total ion intensity was calculated as the sum of the intensities of all relevant ion species specific to poly(HEMA) and the derivatized surface layer. No detection of a peak characteristic of allophanate was observed. Uncoated poly(HEMA) substrate was used as a control for comparison.

The relative normalized peak intensities of various representative negative molecular ions originating from surface layer during various reaction times was determined via TOF-SIMS. The data showed that the major moieties (mostly nitrogen-containing) of the derivatized surface layer appeared during the reaction within 30 min. This was also confirmed by XPS analysis. Total ion intensity was determined as described in the preceding paragraph.

### Example 3

### The Controlled Release of Norfloxacin From a Representative Drug Delivery Construct

C₁₈-methylene chains were coated onto norfloxacin-containing poly(HEMA) for various times (5, 15, 30, and 60 minutes). Initially, it was important to assess the release of norfloxacin from the C₁₈-coated poly(HEMA) in the absence of ultrasound. The data depicted in FIG. 8 demonstrate that, when placed in an aqueous environment, C₁₈-layer has a much lower release rate into the medium, compared to that observed for the uncoated poly(HEMA) control. Furthermore, the initial burst release of the antibiotic was eliminated by the C₁₈-layer. As suggested from the XPS and TOF-SIMS analysis, the progress of the reaction was also confirmed in this experiment. There is little difference in the release of norfloxacin in the material from a 5 minutes reaction vs. the uncoated poly(HEMA), while complete control of antibiotic release is apparent after 30 minutes of reaction time.

### Example 4

### In Vivo Test Results for Representative Intraocular Lens-Hydrogel Constructs: Infection Prevention

In this example, in vivo test results for representative IOL-hydrogel constructs of the invention in infection prevention are described. The IOL-hydrogel constructs were prepared from a hydrogel that included norfloxacin/hydrogel 1% w/w (0.05 mg).

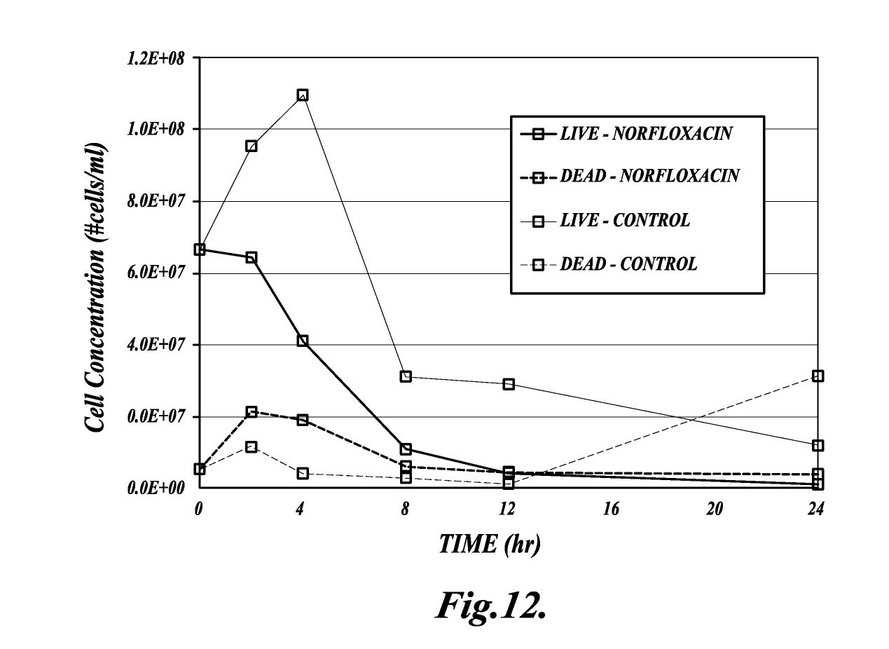

New Zealand white rabbits (n=10) underwent regular lens removal surgery with IOL implantation. FIG. 14 is a photograph showing implantation of the IOL-hydrogel construct.

Post-operatively, the control animals received topical antibiotic drops (norfloxacin eye drop, 2.5 mg/ml, four times a day) and steroids (prednisolone acetate eye drop, 1%, 4 times a day). The experimental animals received only steroid drops (Prednisolone acetate eye drop, 1%, 4 times a day). Aqueous samples from the experimental animals were obtained over time to determine the in vivo antibiotic concentration.

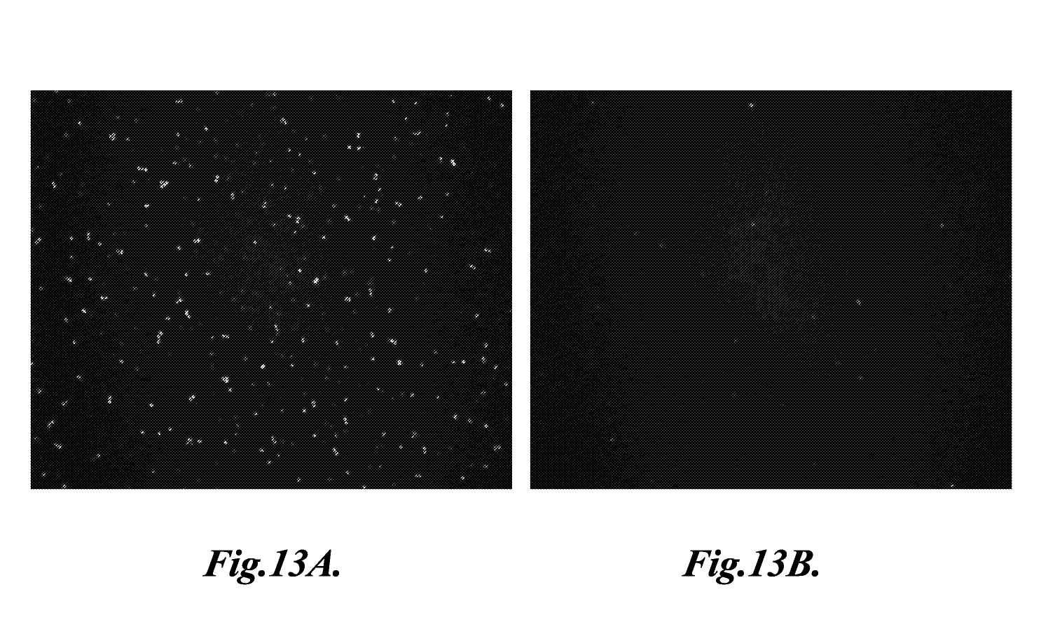

FIGS. 15A (control animal) and 15B (experimental animal) are photographs showing the eye appearance at 20 days post-operation. Referring to FIGS. 15A and 15B, the eyes of the control and experimental animals have similar appearance and do not show clinical infection. The results demonstrate that the IOL-hydrogel construct (releasing antibiotic) is as effective as topical antibiotic administration in preventing and/or treating infection post-cataract removal/IOL implantation surgery.

### Example 5

### In Vivo Test Results for Representative IOL-Hydrogel Constructs: Infection Treatment

In this example, in vivo test results for representative IOL-hydrogel constructs of the invention in infection treatment are described. The IOL-hydrogel constructs were prepared from a hydrogel that included norfloxacin/hydrogel 1% w/w (0.05 mg). The results demonstrate that the IOL-hydrogel constructs of the invention achieve sufficient intra-ocular antibiotic level after cataract surgery to treat severe infection.

Initial in vivo experiments showed that rabbits implanted with a representative IOL-hydrogel construct of the invention did not require additional topical antibiotics after surgery and that the rabbits recovered well. Subsequent tests demonstrated that, when rabbits having implanted IOL-hydrogel constructs of the invention are challenged with active infection, the constructs offer infection control. In this example, the clinical outcomes are compared for rabbits (control, IOL only implanted; and experimental, IOL-hydrogel construct implanted) infected with Staphylococcus epidermidis.

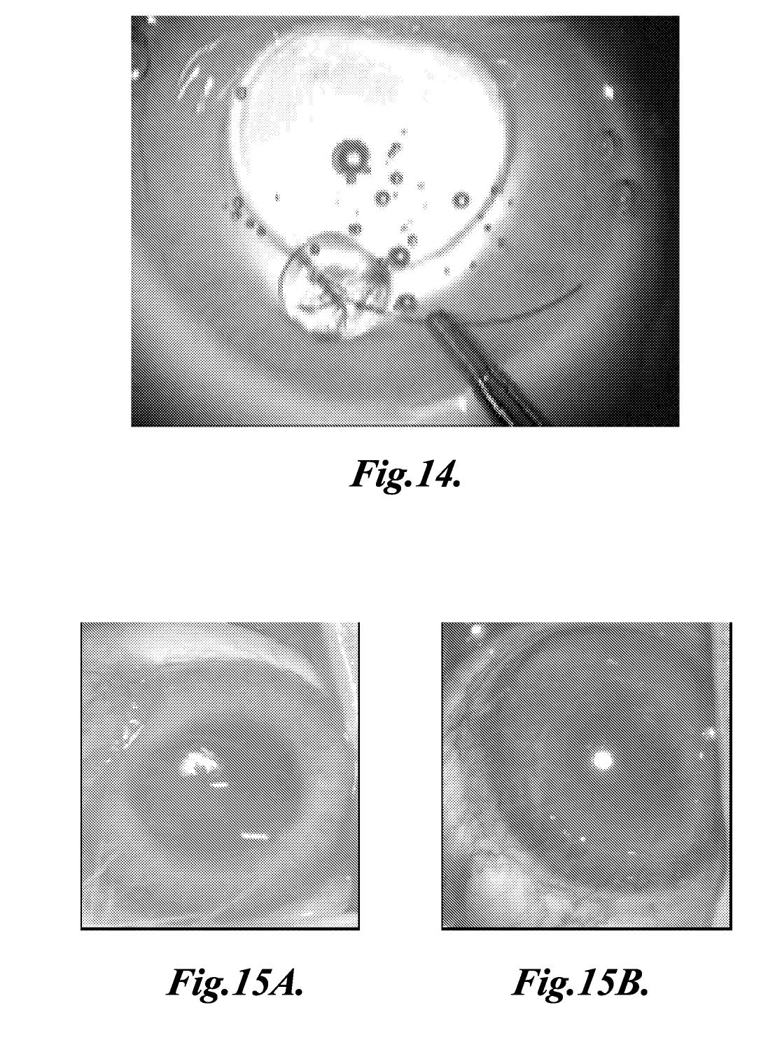

A reliable bacterial endophthalmitis model (Staphylococcus epidermidis or S. epidermidis) was established. The rabbit bacterial challenge protocol was approved by the University of Washington Environmental Health Services. In the model, an optimal dose of S. epidermidis RP62A was established to induce clinical evident endophthalmitis within 24 hours (see FIG. 16). Endophthalmitis was induced after inoculation with 5×10⁴ cfu S. epidermidis.

With in vivo rabbit endophthalmitis model established, in vivo hydrogel testing with bacterial challenge was studied. Each group of the rabbits (n=3) underwent standard cataract surgery with IOL implant (control group, IOL only, no IOL-hydrogel construct; experimental group, IOL-hydrogel construct). Both groups received bacterial challenge on day 1 post-operation. The control group continued to receive topical antibiotics (norfloxacin eye drop, 2.5 mg/ml, four times a day) and steroids (prednisolone acetate eye drop, 1%, 4 times a day), and the hydrogel group only received topical steroids (prednisolone acetate eye drop, 1%, 4 times a day). Both groups of the rabbits developed endophthalmitis after inoculation.

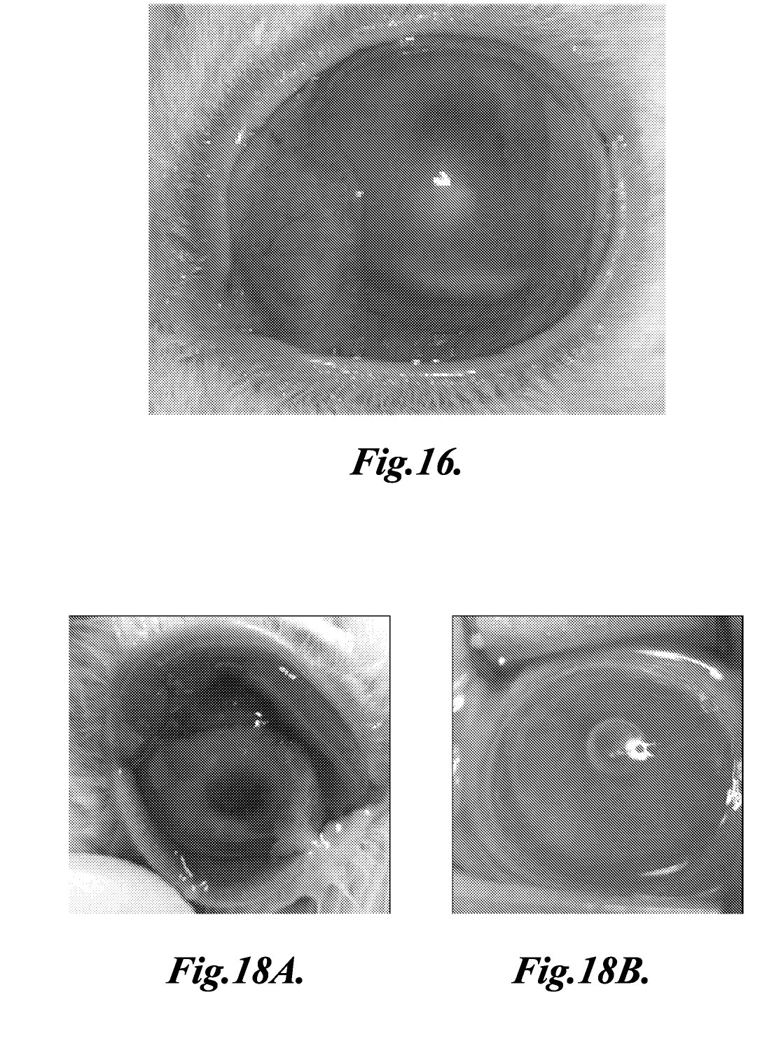

The experimental group recovered from the infection within 3-5 days without additional antibiotics. The control group developed severe infection and the experiment was stopped after day 3. The in vivo antibiotic concentration in the experimental group showed continued higher level drug level compared to MIC (minimum inhibitory concentration) (see FIG. 17). Samples of the aqueous fluid from the rabbit eye was obtained and spectrophotometry analysis of these samples were used to determine the concentration based on previously established calibration curve.

The hydrogel constructs for these rabbits are identical and the in vivo release pattern therefore is very similar. “No challenge” means no SE challenge. The in vivo antibiotic concentration shows similar effective concentration of the antibiotic level under routine and SE challenged conditions—this may explain the fact the rabbits in the infection model recovers.

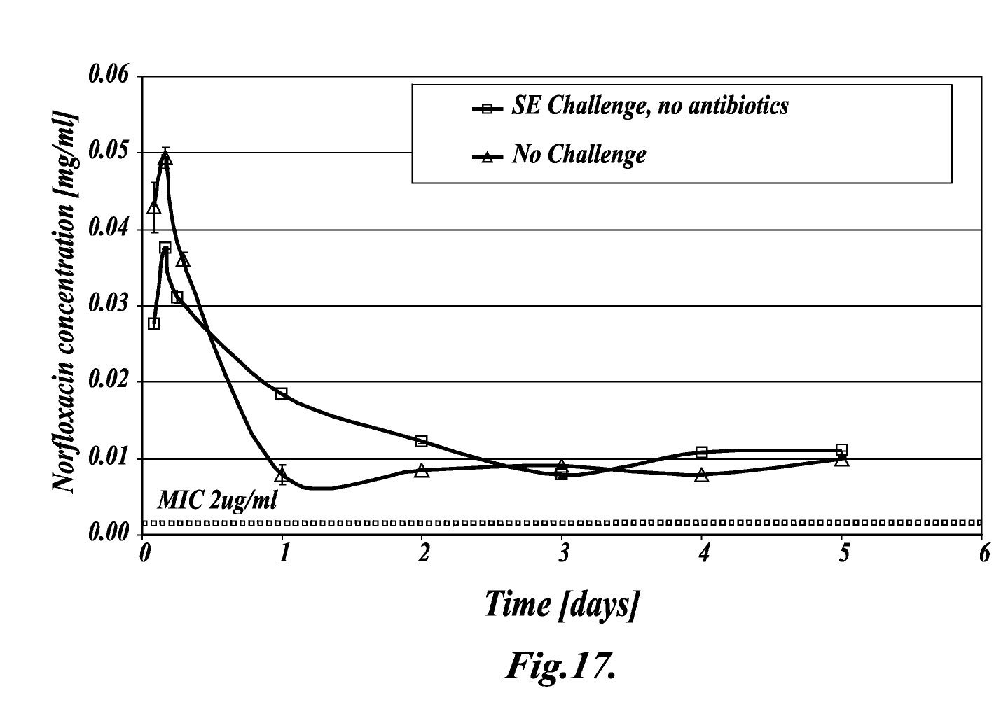

The outcomes of the bacterial challenged rabbits are illustrated in FIGS. 18A (control) and 18B (experimental). FIG. 18A shows a seriously infected eye with discharge and inflammation, a negative outcome for eye infection. FIG. 18B shows the inflammatory reaction to the bacterial challenge, but no full clinical development of severe eye infection.

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. An intraocular device having one or more drug delivery constructs attached thereto, each drug delivery construct comprising a polymeric or hydrogel substrate having a surface with a plurality of alkyl groups covalently coupled thereto, wherein the construct includes a therapeutic drug compound.

2. The device of claim 1, wherein the intraocular device is an intraocular lens or a capsular tension ring.

3. The device of claim 1, wherein the alkyl groups are C₁₀ to C₂₂ unbranched alkyl groups.

4. The device of claim 1, wherein alkyl groups are C₁₂ to C₁₈ unbranched alkyl groups.

5. The device of claim 1, wherein the greater the number of alkyl groups the slower the release of the therapeutic drug compound from the construct.

6. The device of claim 1, wherein the substrate comprises poly(2-hydroxyethyl methacrylate).

7. The device of claim 1, wherein the substrate comprises two or more therapeutic drug compounds.

8. The device of claim 1, wherein the therapeutic drug compound is selected from the group consisting of an antibiotic compound, an anti-inflammatory compound, an ophthalmic beta-blocker, a carbonic anhydrase inhibitor, an alpha-agonist, a miotic compound, and a prostaglandin analog.

9. The device of claim 2, wherein the intraocular lens comprises:
(a) an optic means having an anterior and a posterior surface and a periphery;
(b) at least two resilient haptic means, the haptic means having at least one end thereof secured to the optic means with the haptic means extending outwardly from the periphery of the optic means; and
(c) at least two haptic engaging means formed on the posterior surface of the optic means adjacent the periphery thereof for selectively releasably retaining the haptic means in an inwardly flexed position in proximate relation to the periphery of the optic means, the haptic engaging means being positioned for engagement with the haptic means at a position intermediate the ends thereof.

10. The device of claim 2, wherein the capsular tension ring comprises a loop formed of biocompatible material, the loop being operable to generally prevent shrinkage of the capsular bag following implantation therein.

11. The device of claim 1, wherein the therapeutic drug compound is norfloxacin hydrochloride.

12. A method for intraocular drug delivery, comprising inserting the intraocular device of claim 1 into the eye of a subject in need thereof.

13. The method of claim 12, wherein the intraocular device is an intraocular lens or a capsular tension ring.

14. The method of claim 12, wherein the therapeutic drug compound is selected from the group consisting of an antibiotic compound, an anti-inflammatory compound, an ophthalmic beta-blocker, a carbonic anhydrase inhibitor, an alpha-agonist, a miotic compound, and a prostaglandin analog.

15. A method of treating and/or preventing a disease or condition, comprising introducing the intraocular device of claim 1 into the eye of a subject in need thereof.

16. The method of claim 15, wherein the disease or condition is an infection.

17. The method of claim 15, wherein the intraocular device is an intraocular lens or a capsular tension ring.

18. The method of claim 15, wherein the therapeutic drug compound is selected from the group consisting of an antibiotic compound, an anti-inflammatory compound, an ophthalmic beta-blocker, a carbonic anhydrase inhibitor, an alpha-agonist, a miotic compound, and a prostaglandin analog.

19. A kit for attaching a drug delivery construct to an intraocular device, comprising:
(a) a tube with one or more drug delivery constructs attached thereto, each drug delivery construct comprising a polymeric or hydrogel substrate having a surface with a plurality of alkyl groups covalently coupled thereto, wherein the construct includes a therapeutic drug compound; and
(b) a tool for removing the drug delivery constructs from the tube to an intraocular device.
